(19) United States
(12) Patent Application Publication
(10) Pub. No.: US 2006/0047296 A1
(43) Pub. Date: Mar. 2, 2006

(54) Annulus replacement system and technique

(75) Inventors: Jill Embry, Memphis, TN (US); Joseph Pizzurro, Collierville, TN (US); Randy Allard, Germantown, TN (US); Robert Rice, Southaven, MS (US)

Correspondence Address: HAYNES AND BOONE, LLP, 901 MAIN STREET, SUITE 3100, DALLAS, TX, 75202, US

(73) Assignee: SDG Holdings, Inc., Wilmington, DE (US)

(21) Appl. No.: 10/930,580

(22) Filed: Aug. 31, 2004

(51) Int. Cl. A61M 29/00

(52) U.S. Cl. 606/191

(57) ABSTRACT

An annulus replacement technique includes inserting a guide wire into an annulus space. A first dilator tube is then slid over the guide wire and into the intervertebral space. The first dilator tube has a first longitudinal channel of a first cross section that is shaped to matingly engage with the guide wire. A second dilator tube is then slid over the first dilator tube and into the intervertebral space. The second dilator tube has a second longitudinal channel of a second cross section that mating engages with an outer cross section of the first dilator tube. The first dilator tube and the guide wire can then be removed and an implant can be inserted into the intervertebral space through the second dilator tube.

### BACKGROUND

In the treatment of diseases, injuries or malformations affecting spinal movement and disc tissue, it has long been common practice to remove a portion or all of a degenerated, ruptured, or otherwise failing intervertebral disc thereby leaving an intervertebral space. The intervertebral disc serves as a cushion between the vertebral bodies so as to permit controlled motion. A healthy intervertebral disc consists of three components: a gelatinous inner core called the nucleus pulposus (or, more simply, the nucleus); a series of overlapping and laminated plies of tough fibrous rings called the annulus fibrosus (or, more simply, the annulus); and two (i.e., superior and inferior) thin cartilage layers, connecting the intervertebral disc to the thin cortical bone of the adjacent vertebral bodies, called the end plates. When a portion or all of the intervertebral disc is removed, something often needs to be done with the corresponding space. Typically, an implant is provided to fill the space. The implant can be of the type that promotes fusion between the adjacent vertebral bodies. Alternatively, the implant can be of the type that allows movement or some other function that would exist in a normal anatomical structure.

Modern trends in surgery include the restoration of the intervertebral space using minimally invasive surgical techniques. The ability to surgically repair damaged tissues or joints, creating as few and as small incisions as possible, generally produces less trauma and pain for the patient while yielding better clinical outcomes.

### SUMMARY

The present invention provides a new and improved system, kit, assembly, and method for restoring an intervertebral space using minimally invasive surgical techniques. In one embodiment, the method includes inserting a guide wire into the intervertebral space, such as an annulus space. A first dilator tube is then slid over the guide wire and into the intervertebral space. The first dilator tube has a first longitudinal channel of a first cross section that is shaped to matingly engage with the guide wire. A second dilator tube is then slid over the first dilator tube and into the intervertebral space. The second dilator tube has a second longitudinal channel of a second cross section that mating engages with an outer cross section of the first dilator tube. The first dilator tube and the guide wire can then be removed and an implant can be inserted into the intervertebral space through the second dilator tube.

In some embodiments, the first and second dilator tubes have a circular cross section. Also, additional dilator tubes can be utilized to further increase the retraction and separation of the intervertebral space.

In some embodiments, the implant is disposed in an implant tube having an outer cross section similar to that of the outer cross section of the first dilator tube. In these embodiments, the step of inserting the implant includes inserting the implant tube.

In some embodiments, the implant is an annulus replacement implant. The implant may include a suture having two ends. Both ends of the suture can extend from the a proximal end of the implant tube. The implant may also include a porcine pericardium that is woven with the suture.

In embodiments including a suture, a knot can be tied in the two suture ends to form the implant into a desired shape. Further, the knot can be pressed towards the intervertebral space using a pressing member pushed through the second dilator tube.

In another embodiment of the invention, an annulus replacement implant assembly is provided. The annulus replacement implant assembly includes a hollow tube having an outer circumference that corresponds with an inner circumference of a cannula inserted into a retracted intervertebral space. The annulus replacement implant assembly also includes a suture and an implant woven around the suture and positioned with the suture inside the hollow tube. In some embodiments, the implant is formed of pericardium.

In another embodiment of the invention, a surgical kit is provided. The surgical kit includes a first retractor tube having a tapered tip for inserting into a space, a second retractor tube having an interior cross section that is of a shape for slidably engaging with an exterior of the first retractor tube, a third retractor tube having an interior cross section that is of a shape for slidably engaging with an exterior of the second retractor tube, and a fourth retractor tube having an interior cross section that is of a shape for slidably engaging with an exterior of the third retractor tube. The kit further includes a first implant tube having an exterior cross section that is of a shape for slidably engaging with the interior cross section of the fourth retractor tube.

In some embodiments, the implant tube includes an annulus replacement implant made of porcine pericardium and a suture woven around the annulus replacement implant.

In another embodiment of the invention, another surgical kit is provided. The surgical kit includes a guide wire shaped and configured for percutaneously inserting into an intervertebral space from a lateral approach. The kit also includes a first dilator tube having a first longitudinal channel and a tapered distal end for retracting the intervertebral disc space and a second dilator tube having a second longitudinal channel and a tapered distal end for retracting the intervertebral disc space. The guide wire, the first dilator tube, and the second dilator tube are configured for telescopic interaction about a common longitudinal axis. The kit further includes an implant tube configured for temporarily containing an intervertebral implant and for being insertable into the intervertebral space through the second dilator tube

In some embodiments of the kit, the first and second dilator tubes have a circular cross section.

In some embodiments, the kit includes a handle assembly for engaging with the second dilator tube. The handle assembly includes a hand piece and a locking mechanism for selectively engaging and securing the hand piece with the second dilator tube.

Additional embodiments are provided in the detailed specification above. None of the elements and features of the above and below described embodiments are considered essential to the invention, and the invention should be construed consistent with the claims listed at the end of the present disclosure.

### BRIEF DESCRIPTION OF THE DRAWINGS

FIG. 1 is a side view of vertebral column having a damaged or diseased annulus and a corresponding intervertebral disc space.

FIG. 2 is a flowchart of a method for replacing an annulus in the vertebral column of FIG. 1.

FIG. 3 is a top view of a guide wire and retractor system for retracting the disc space and for inserting an implant according to one embodiment of the present invention.

FIG. 4 is a top view of an implant tube including an implant being inserted into the retracted space created as discussed in FIG. 3.

FIG. 5 is a top view of the implant tube and implant of FIG. 4.

FIGS. 6a and 6b are top views of the implant tube of FIG. 3 with a handle and bolt used for further inserting the tube into the retracted space.

FIGS. 7a-7f are illustrations of the bolt discussed with respect to FIGS. 6a, 6b.

FIGS. 8a-8c are illustrations of the handle discussed with respect to FIGS. 6a, 6b.

FIG. 9 is a top view of a combination braid inserter/knot tightener used to insert the implant into the annulus space.

FIGS. 10a-10c are various views of the combination braid inserter/knot tightener discussed with respect to FIG. 9.

FIGS. 11a-11b are top and cross-sectional views, respectively, for illustrating how a knot in a suture of the implant is tied using the combination braid inserter/knot tightener of FIGS. 10a-10c.

FIG. 12 is a picture of the implant that has been formed and shaped by one or more knots.

### DETAILED DESCRIPTION

The present disclosure relates generally to the field of orthopedic surgery, and more particularly to the instrumentation and techniques for inserting intervertebral implants. For the purposes of promoting an understanding of the principles of the invention, reference will now be made to embodiments or examples illustrated in the drawings, and specific language will be used to describe the same. It will nevertheless be understood that no limitation of the scope of the invention is thereby intended. Any alteration and further modifications in the described embodiments, and any further applications of the principles of the invention as described herein are contemplated as would normally occur to one skilled in the art to which the invention relates. To facilitate the understanding of the invention, many of the drawings are out of scale, or include components that are out of scale. In addition, certain steps may not be shown in detail in the drawings, but are described below and are generally well known in the art. Furthermore, although reference numbers are repeated throughout the embodiments, this does not by itself dictate a relationship between the elements, but is instead provided for the sake of example.

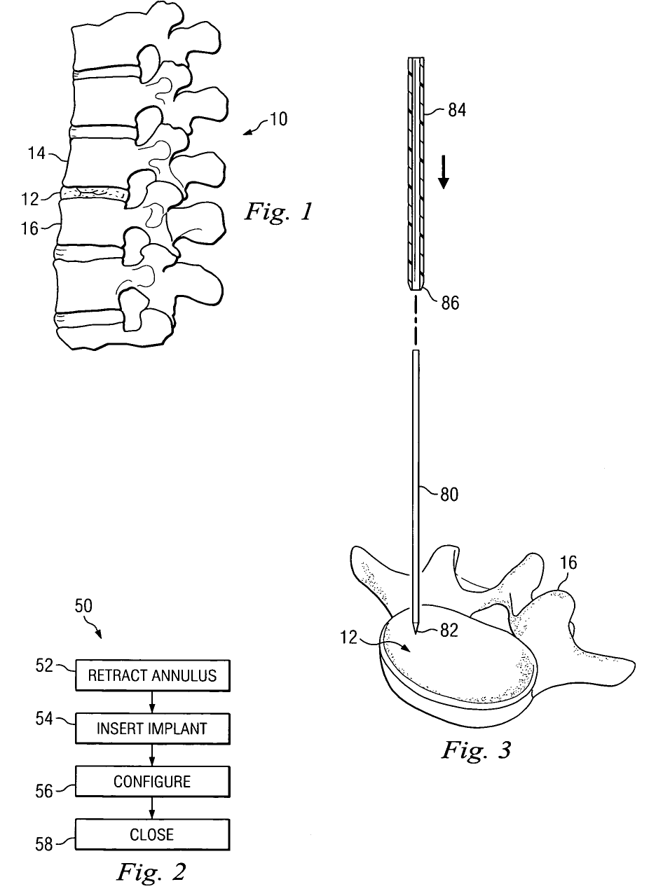

Referring first to FIG. 1, the numeral 10 refers to vertebral column with a damaged or diseased annulus 12 extending between vertebrae 14 and 16. In the present embodiments, the annulus 12 is removed to form an annulus space, and an annulus replacement is inserted into the annulus space.

Referring now to FIG. 2, a method 50 can be used for replacing the annulus 12 or portions thereof. It is understood that the method 50 can be used with other methods and surgical techniques. For example, a nucleus replacement technique can be performed before, during, and/or after the method 50, as desired. The present method 50 is discussed in general detail below, and then several embodiments of instruments and implants for use with the method are discussed in greater detail with respect to the remaining figures of the present disclosure.

The method begins at step 52 wherein an approach to the annulus 12, and the corresponding disc space, is created. In the present embodiment, the approach is created via a minimally invasive lateral opening using a cannula. The cannula produces a passageway of sufficient size with which to insert and configure an annulus replacement implant. It is understood that if other procedures are being performed, the approach can be combined with the other procedure, or can use the same approach as the other procedure, as needed (e.g., an anterior approach, a posterior approach, and so forth). In some embodiments, existing tissue may need to be moved or removed to facilitate the insertion of the annulus replacement implant. Removal can be specific to the diseased or damaged annulus, although in other embodiments an entire discectomy can be performed.

At step 54, the annulus replacement implant is provided via an implant tube through the previously created passageway. The annulus replacement implant is positioned in the perimeter of the disc space, corresponding to a location of a normal, natural annulus if one were to have existed.

At step 56, the annulus replacement implant is positioned and formed into a desired configuration. In one embodiment, the implant includes a suture which can receive one or more knots. The sutures can be manipulated to form or “crunch” the implant into the desired configuration and once in that configuration, the sutures can be tied off into a knot.

At step 58, the remaining instruments and other material are removed as needed, and the passageway is closed. In the above-mentioned embodiment, the sutures are cut off and the cannula is removed.

Referring now to FIGS. 3-12b, additional embodiments are provided for performing one or more of the method steps discussed above. It is understood that the embodiments provided may be combined with other procedural steps, and that some embodiments may not be used in some procedures.

Referring to FIG. 3, a guide wire 80 is inserted via a lateral or posterio-lateral approach (collectively a lateral approach) into the annulus space 12 of the vertebrae 16. In furtherance of the present embodiment, the guide wire 80 is relatively narrow yet rigid, and includes a diamond-beveled tip 82 for performing a percutaneous insertion to the annulus space 12.

Once the guide wire 80 is in place, a first dilator tube 84 is inserted coaxially with a longitudinal axis of the guide wire. The first dilator tube 84 includes a tapered tip 86 for assistance in insertion and dilation of a passageway originally formed by the guide wire 80. Also, the first dilator tube 84 has an inside diameter that is substantially equal to or slightly greater than an outside diameter of the guide wire 80, so that the first dilator tube 84 slides snugly over the guide wire. In the present embodiments, the guide wire 80 and the first dilator tube 84 have a circular cross section. It is understood that in other embodiments, one or more of the guide wire and/or dilator tubes can have different shaped cross sections, such as oval, triangular, square, rectangular, and so forth. For example, the guide wire can have a triangular cross section and the first dilator tube can have a circular inside cross section that matingly engages with the triangular cross section of the guide wire.

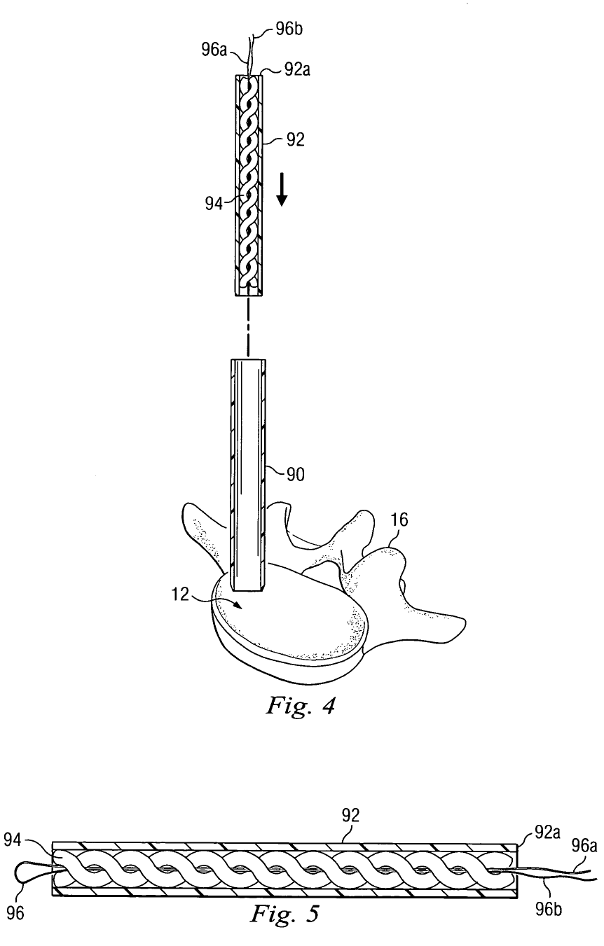

The first dilator tube 84 is inserted into the annulus space 12. At this time, the guide wire 80 can be removed, if desired. A second dilator tube can then be inserted in a manner similar to the first dilator tube 84. The second dilator tube can be constructed similarly to the first dilator tube 84, except that an inside cross section of the second dilator tube is substantially equal to or slightly greater than an outside cross section of the first dilator tube. After this, a third dilator tube (FIG. 4) can be inserted in a manner similar to that of the second dilator tube, thereby sequentially increasing the passageway into the annulus space 12. This process is similar, at least in part, to the process described in U.S. Pat. No. 5,752,969, which is hereby incorporated by reference as if reproduced in its entirety.

It is understood that the number of dilator tubes used can be dependent on several variables. For one embodiment, each of the dilator tubes can be relatively thin walled. In this embodiment, many dilator tubes can be used with each dilator tube only dilating a relatively small amount. This embodiment provides a very gradual and controlled dilation. In some embodiments, the final dilation may vary dependent on various conditions. For example, a kit can be provided with many (e.g., eight) dilator tubes of increasing diameter. The kit may also provide several different sized implant tubes (FIGS. 4 and 5). The dilator tubes can then be inserted and a choice can be made as to what amount of dilation is ultimately desired, and therefore how many of the dilator tubes will be used. For example, a smaller person may require only a comparatively small amount of dilation and therefore only two or three of the dilation tubes can be used. A larger person may require more dilation and seven or eight of the dilation tubes can be used. Also, the amount of dilation can be determined as the process is performed, responsive to the reaction of the patient to the surgery. Furthermore, the amount of dilation can be determined based on the final annulus space 12 that is created, which may be unknown until midway through the surgery.

It is also understood that in the present embodiments, the guide wire 80 and the dilator tubes 84 (et al.) include tapered ends and are formed of a material that is of sufficient strength to perform retraction. The strength of the retractor tubes may be a combination of material used, e.g., metal or quartz, and/or a cross-sectional thickness of the tubes. Also, some shapes, such as circular, can provide additional structural strength for retraction. In applications other than the annulus space 12, such as in parts of the body away from the spine, thinner tubes can be used if a smaller retraction force is sufficient.

Referring to FIG. 4, the last dilator tube is designated with a reference numeral 90. The guide wire and other dilator tubes can be removed so that the last dilator tube 90 serves as a cannula for one or more of the following steps. Referring also to FIG. 5, once the last dilator tube/cannula 90 is in place, an implant tube 92 can be inserted therein. The implant tube 92 includes an annulus replacement implant 94 which will be inserted into the annulus space 12.

Although several different annulus replacement implants can be used, in the present embodiment, the implant 94 is a braided formation of porcine pericardium. The pericardium is braided around a suture 96 and is slideable about the suture. In the present embodiment, the implant 94 comes prepackaged inside the implant tube 92 and both ends of the suture 96 extend out of a common end 92a of the implant tube 92. In this way, a remote facility (e.g., a laboratory) can package the implant 94 into the implant tube 92, seal the tube (if desired), and then deliver it to the operating room.

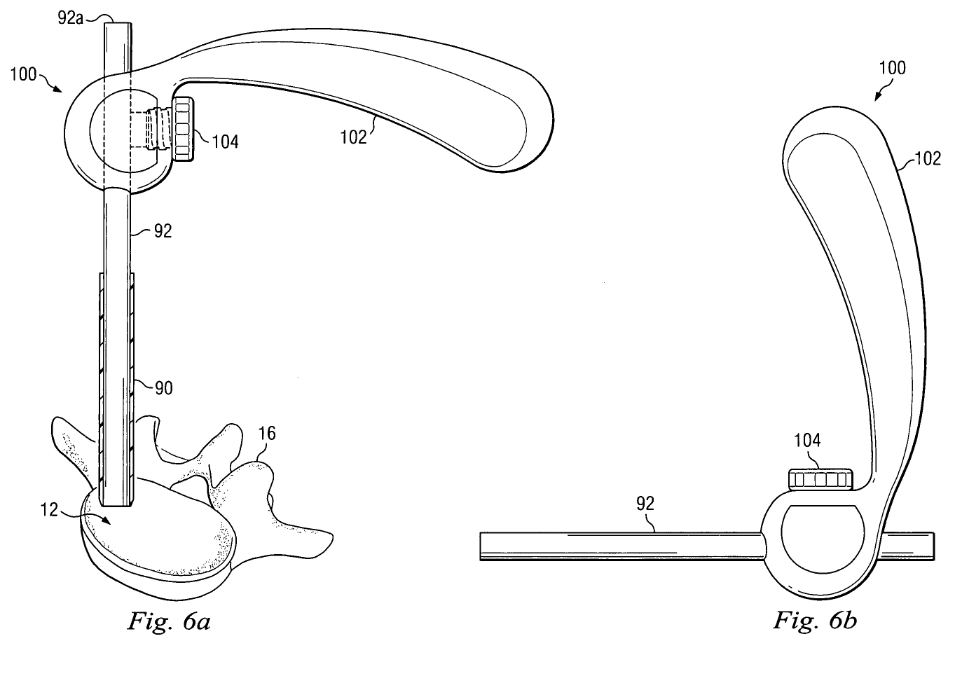

Referring now to FIGS. 6a-6b, with the implant tube 92 positioned inside the cannula 90, a handle assembly 100 can be attached near the end 92a of the implant tube to facilitate and secure the final and exact insertion position of the implant tube into the annulus space 12. The handle assembly 100 includes a handle portion 102 and a drive-bolt assembly 104.

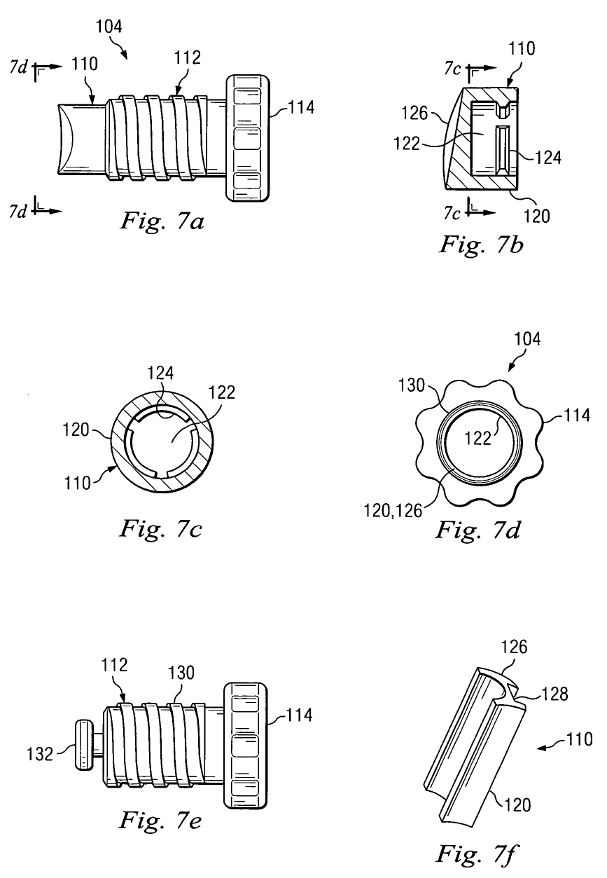

Referring to FIGS. 7a-7f, the drive bolt assembly 104 includes a drive member 110, a threaded member 112, and a head 114. Referring specifically to FIGS. 7b-7c, in one embodiment, the drive member 110 includes a body 120 surrounding a cavity 122 and including a plurality of flanges 124. A proximal surface 126 of the drive member 110 conforms to the exterior shape of the implant tube 92 and can therefore secure the implant tube inside the handle assembly 100. The flanges 124 are provided so that the drive member 110 can engage with the threaded member 112 without having to rotate with the threaded member, as discussed in greater detail below.

Referring to FIG. 7f, in another embodiment, the drive member 110 is a shaped relatively long, as compared to the threaded member 112. The drive member 110 includes a groove 128 for engaging with the threaded member 112. The groove 128 performs like the flanges 124 in the previous embodiment so that the drive member 110 can engage with the threaded member 112 without having to rotate with the threaded member, as discussed in greater detail below.

Referring to FIG. 7e, the threaded member 112 includes threads 130 for engaging with the handle portion 102 and a protrusion 132 for engaging with the flanges 124 and/or groove 128 of the drive member 110. The threaded member is securely attached to the head 114 so that the threaded member can be rotated, and hence advanced, by rotation of the head.

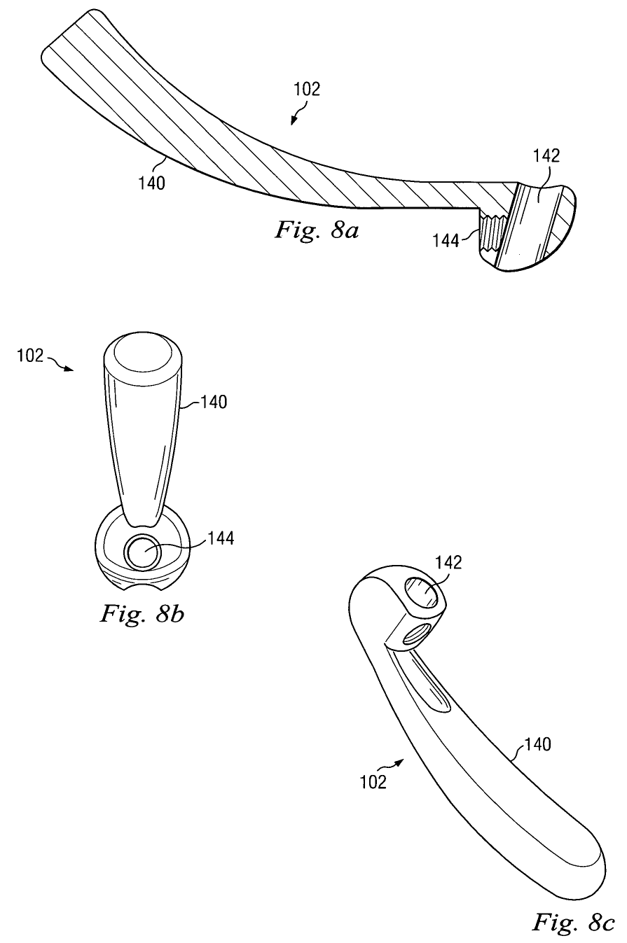

Referring now to FIGS. 8a-8c, the handle portion 102 includes a gripping lever 140, a tube aperture 142, and a locking aperture 144. The tube aperture 142 is sized and shaped to fit snuggly around the outside of the implant tube 92. In the present embodiment, the implant tube 92 has a round diameter and is therefore able to rotate inside the tube aperture. In embodiments where the implant tube 92 is oval or has a non-circular shape, the inherent shape of the implant tube will secure the tube from rotating inside the tube aperture 142. Some of these embodiments may not utilize a drive bolt assembly. In continuation of the present embodiment where the implant tube 92 is circular, the implant tube is secured in the tube aperture 142 by inserting the drive-bolt assembly 104 into the locking aperture 144. The locking aperture has threads that correspond to the threads 130 of the drive bolt assembly 104 (FIG. 7e). In this way, the drive-bolt assembly 104 can be tightened down through the locking aperture 144 to force the surface 126 to engage with, and thereby secure, the implant tube 92 to the handle portion 102.

Once the implant tube 92 is secured to the handle assembly 100, the implant tube can be further inserted or removed (as needed), and can be secured in position while further steps are taken.

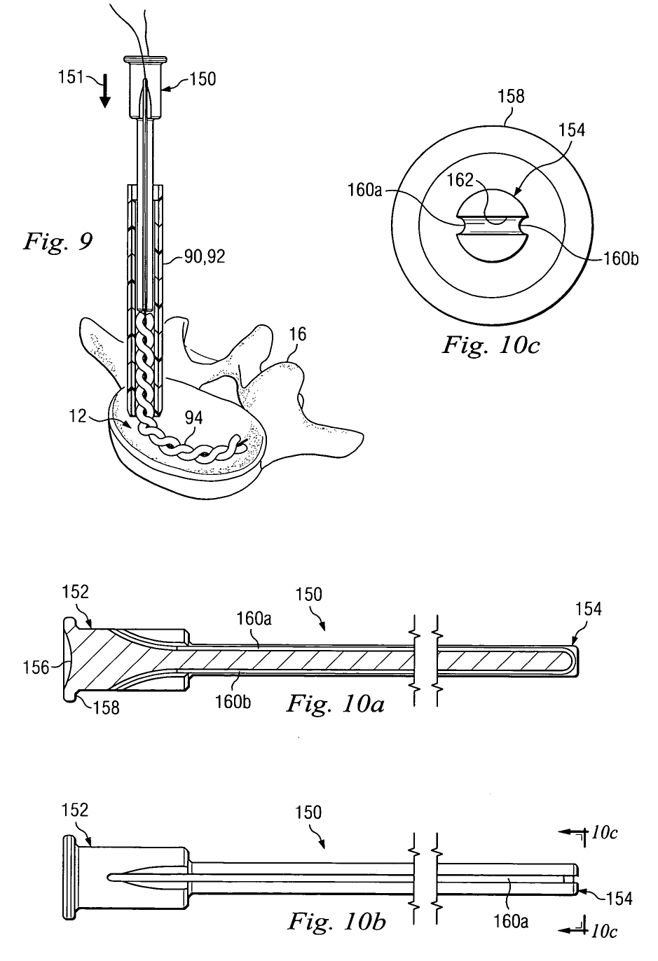

Referring now to FIG. 9, with the implant tube 92 positioned inside the cannula 90, a combination braid inserter/knot tightener 150 can be inserted in the proximal end 92a of the implant tube 92. Pressure in a direction 151 can be applied to the combination braid inserter/knot tightener 150 to force the annulus replacement implant 94 out of the implant tube 92 and into the annulus space 12. The annulus replacement implant 94 forms the desired shape by one or more factors. The previously-created annulus space 12 can be used to shape the annulus replacement implant 94 into a ring, similar to a natural annulus. Also, the annulus replacement implant 94 can be formed and shaped by using the sutures 96, discussed in greater detail below.

Referring now to FIGS. 10a-10c, the combination braid inserter/knot tightener 150 includes a proximal gripping end 152 and a distal braid-interacting end 154. The gripping end 152 includes an indention 156 by which a surgeon can press his/her thumb to apply the pressure in the direction 151 (FIG. 9). The gripping end 152 also includes a lip 158 by which the combination braid inserter/knot tightener 150 can be held firmly in place and can be removed from the implant tube 92.

Referring specifically to FIGS. 10b and 10c, the combination braid inserter/knot tightener 150 also includes a two longitudinal grooves 160a, 160b that extend along the length of the device towards the distal braid-interacting end 154. The distal braid-interacting end 154 also includes an indention 162 between the grooves 160a, 160b. The grooves 160a, 160b are of sufficient size that the two ends 96a, 96b of the suture 96 (FIGS. 4 and 5) can be placed in the grooves and the combination braid inserter/knot tightener 150 can still be moved through the implant tube 92.

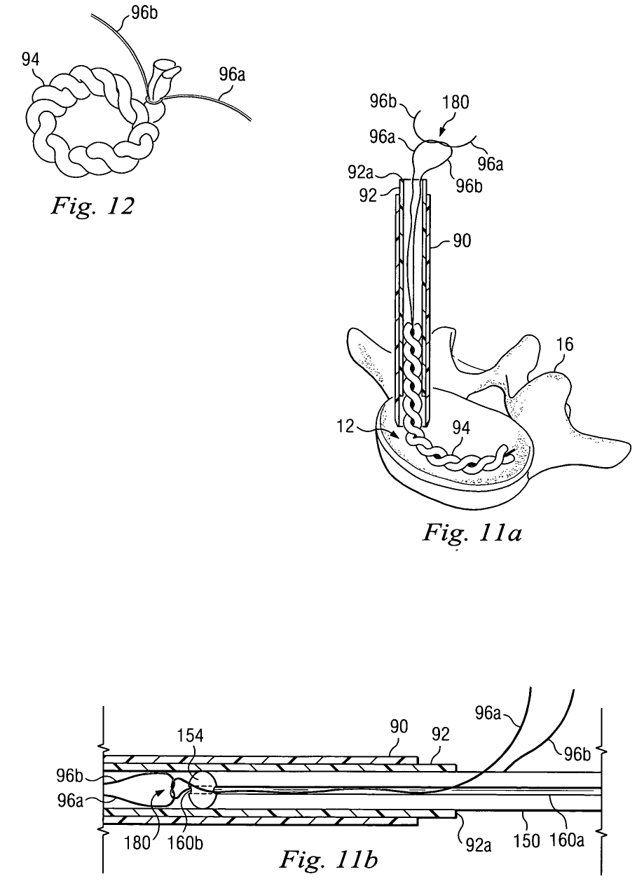

Referring now to FIG. 11a, once the annulus replacement implant 94 is inserted into the annulus space 12 (either partially or fully, as desired), the combination braid inserter/knot tightener 150 is removed from the implant tube 92. As shown in FIG. 11a, the ends 96a, 96b of the suture remain extending from the end 92a of the implant tube 92. The ends 96a, 96b can then be tied into a knot 180 at a distance from the annulus space 12. By tying and later tightening the knot 180, the implant 94 effectively “crumples” and forms into the desired shape of the annulus space 12.

Referring to FIG. 11b, the combination braid inserter/knot tightener 150 can then be positioned so that the end 154 is against the knot 180 and the grooves 160a, 160b are aligned with the suture ends 96a, 96b, respectively. The combination braid inserter/knot tightener 150 can then be used to press the knot 180 down the implant tube 92 and towards the annulus replacement implant 94. This serves to tighten the knot 180 and further facilitates the “crumpling” and forming of the implant 94 into the desired shape.

Subsequent knots 180 can be added to continue the crumpling and forming of the implant 94 and securing the implant into its final form. For example, the implant 94 can be partially inserted into the annulus space 12, the knot 180 can be formed and partially tightened with the combination braid inserter/knot tightener 150. The combination braid inserter/knot tightener 150 can then be used to further insert the implant 94, further tighten the knot 180, and further form the implant until it reaches its final desired form and position.

After the implant 94 is in place and the knot(s) 180 have been tied, the combination braid inserter/knot tightener 150 can be removed, the implant tube 92 can be removed, and the suture ends 96a, 96b can be cut to a desired length. The cutting can be performed through the cannula 90. The cannula 90 can then be removed and the surgical sight can be closed using ordinary procedures.

Although only a few exemplary embodiments of this invention have been described in detail above, those skilled in the art will readily appreciate that many modifications are possible in the exemplary embodiments without materially departing from the novel teachings and advantages of this invention. For example, although the teachings have been directed to the intervertebral space, other embodiments of the present invention can be directed to other areas, including the knee, the femur, and so forth. Accordingly, all such modifications are intended to be included within the scope of this invention as defined in the following claims.

What is claimed is:

1. A surgical method sequentially comprising:
inserting a guide wire into an intervertebral space;
sliding a first dilator tube over the guide wire and into the intervertebral space, the first dilator tube having a first longitudinal channel of a first cross section that is shaped to matingly engage with the guide wire;
sliding a second dilator tube over the first dilator tube and into the intervertebral space, the second dilator tube having a second longitudinal channel of a second cross section that matingly engages with an outer cross section of the first dilator tube;
removing the first dilator tube and the guide wire; and
inserting an annulus replacement implant into the intervertebral space through the second dilator tube.

2. The surgical method of claim 1 wherein the implant comprises a suture having two ends, and wherein the two ends of the suture extend from the a proximal end of the implant tube.

3. A surgical method sequentially comprising:
inserting a guide wire into an intervertebral space;
sliding a first dilator tube over the guide wire and into the intervertebral space, the first dilator tube having a first longitudinal channel of a first cross section that is shaped to matingly engage with the guide wire;
sliding a second dilator tube over the first dilator tube and into the intervertebral space, the second dilator tube having a second longitudinal channel of a second cross section that matingly engages with an outer cross section of the first dilator tube;
removing the first dilator tube and the guide wire; and
inserting an implant into the intervertebral space through the second dilator tube;
wherein the implant is disposed in an implant tube having an outer cross section similar to that of the outer cross section of the first dilator tube, and the step of inserting the implant includes inserting the implant tube.

4. The surgical method of claim 3 wherein the implant is an annulus replacement implant.

5. The surgical method of claim 4 wherein the first and second dilator tubes have a circular cross section.

6. The surgical method of claim 4 wherein the implant comprises a suture having two ends extending from the a proximal end of the surgical method further comprising:
tying a knot in the two suture ends to form the implant into a desired shape.

7. The surgical method of claim 6 further comprising:
pressing the knot towards the intervertebral space using a pressing member pushed through the second dilator tube.

8. The surgical method of claim 4 wherein the implant comprises porcine pericardium.

9. The surgical method of claim 3 further comprising:
securing the second dilator tube prior to inserting the implant.

10. An annulus replacement implant assembly comprising:
a hollow tube having an outer circumference that corresponds with an inner circumference of a cannula inserted into a retracted intervertebral space a suture; and
an implant woven around the suture and positioned with the suture inside the hollow tube.

11. The annulus replacement implant assembly of claim 10 wherein the implant is formed of pericardium.

12. The annulus replacement implant assembly of claim 10 wherein the implant includes porcine pericardium.

13. A surgical kit comprising:
a first retractor tube having and a tapered tip for inserting into a retraction space;
a second retractor tube having an interior cross section that is of a shape for slidably engaging with an exterior of the first retractor tube;
a third retractor tube having an interior cross section that is of a shape for slidably engaging with an exterior of the second retractor tube;
a fourth retractor tube having an interior cross section that is of a shape for slidably engaging with an exterior of the third retractor tube; and
a first implant tube having an exterior cross section that is of a shape for slidably engaging with the interior cross section of the fourth retractor tube.

14. The kit of claim 13 further comprising:
an implant extractor having an exterior cross section that is of a shape for slidably engaging with an interior cross section of the implant tube.

15. The kit of claim 13 wherein the implant tube includes an annulus replacement implant and a suture woven around annulus replacement implant.

16. The kit of claim 15 wherein the implant is formed of porcine pericardium.

17. The kit of claim 13 further comprising:
a second implant tube having an exterior cross section that is of a shape for slidably engaging with the interior cross section of the third retractor tube.

18. A surgical kit comprising:
a guide wire shaped and configured for percutaneously inserting into an intervertebral space from a lateral approach;
a first dilator tube having a first longitudinal channel and a tapered distal end for retracting the intervertebral disc space;
a second dilator tube having a second longitudinal channel and a tapered distal end for retracting the intervertebral disc space; and
an implant tube configured for temporarily containing an intervertebral implant and for being insertable into the intervertebral space through the second dilator tube;
wherein the guide wire, the first dilator tube, and the second dilator tube are configured for telescopic interaction about a common longitudinal axis.

19. The surgical kit of claim 18 wherein the first and second dilator tubes have a circular cross section.

20. The surgical kit of claim 18 further comprising:
a handle assembly for engaging with the second dilator tube, the handle assembly includes a hand piece and a locking mechanism for selectively engaging and securing the hand piece with the second dilator tube.

21. The surgical kit of claim 20 wherein the locking mechanism includes a handle portion rigidly connected to a threaded portion rotationally connected to a retaining portion, wherein the threaded portion corresponds to mating threads in the hand piece, and wherein the retaining portion is configured to engage with the second dilator tube by pressing, in a non-rotational manner, against the second dilator tube in response to a rotation of the threaded portion.
